(19) United States
(12) Patent Application Publication
(10) Pub. No.: US 2011/0307056 A1
(43) Pub. Date: Dec. 15, 2011

(54) MEDICAL VALVE IMPLANT FOR IMPLANTATION IN AN ANIMAL BODY AND/OR HUMAN BODY

(75) Inventor: Nils Le Cerf, Zollikon (CH)

(73) Assignee: BIOTRONIK AG, Bulach (CH)

(21) Appl. No.: 13/110,267

(22) Filed: May 18, 2011

Related U.S. Application Data
(60) Provisional application No. 61/352,835, filed on Jun. 9, 2010.

(51) Int. Cl. A61F 2/24

(52) U.S. Cl. 623/2.17; 623/2.19

(57) ABSTRACT

The invention relates to a medical valve implant for implantation in the animal body and/or human body, comprising a support frame that includes a first end and a second end, which are disposed on opposite ends of the support frame in a main extension direction of support frame. According to the invention, one or more auxiliary devices that are required for implantation and/or that serve a functional purpose for a limited time and are intended to remain in the body after implantation are formed, at least in sections, of a material that can decompose in the body.

### CROSS-REFERENCE TO RELATED APPLICATIONS

This invention claims benefit of priority to U.S. patent application Ser. No. 61/352,835, filed Jun. 9, 2010; the contents of which are herein incorporated by reference in their entirety.

### TECHNICAL FIELD

The invention relates to a medical valve implant for implantation in an animal body and/or human body, and more specifically to a medical valve implant having improved long-term properties.

### BACKGROUND

Implants are used in medical applications that are implanted in an animal body and/or human body permanently or at least for a longer period of time to perform replacement functions. Valve implants are known, for example, such as aortic valve implants that perform the function of the natural aortic valve.

Aortic valves are known, for example, in the case of which the diseased cusp is clamped between the valve implant and the aortic wall, thereby constricting the lumen. A valve implant of this type in the form of an artificial aortic valve is known e.g. from U.S. Pat. No. 7,399,315. The valve implant includes an elastic support frame that carries and supports the valve, and on which metallic positioning bodies are disposed, using which the valve implant is anchored at its intended location in the luminal wall such that it fits with the natural valve. The support frame is covered with material that should support the adhesion of the valve implant.

A problem that occurs frequently with valve implants, particularly in the heart, is calcification i.e. the deposition of calcium salts, in particular calcium phosphate (hydroxyapatite). Calcification can occur in and on some materials of a medical implant while they come in contact with the patient's bodily fluids. Calcification can impair the performance and structural integrity of medical implants composed of calcification-sensitive materials, particularly over longer periods of time. For example, calcification is the main cause of clinical failure of bioprosthetic heart valves made from porcine aortic valves or bovine pericardia. Calcification occurs particularly intensively at stress points at which e.g. a suture passes through tissue. Calcification also greatly affects the performance of valve implants composed of synthetic materials such as polyurethane.

In general, bioprosthetic heart valves begin to fail approximately seven years after implantation, and only a few bioprosthetic heart valves are still functional after 20 years. The need to replace a degenerating valve implant subjects the patient to an additional surgical risk, wherein the old implant must be removed or remain at the site of implantation.

### SUMMARY

A feature of the invention is to create a medical valve implant having improved long-term properties.

A feature is solved according to the invention by the features that follow. Favorable embodiments and advantages of the invention will be appreciated by the skilled artisan in view of the drawings and the description.

The invention is directed to a medical valve implant comprising a support frame that has a first end and a second end that are disposed on opposite ends of the support frame in a main extension direction of the support frame.

According to the invention, one or more auxiliary devices that are required for implantation and/or that serve a functional purpose for a limited time and are intended to remain in the body after implantation are formed, at least in sections, of a material that can decompose in the body. In particular, the one or more auxiliary devices can also be formed entirely of a material that can decompose in the body.

As an alternative or in addition thereto, it is provided that the support frame is formed, at least in sections, of a material that can decompose in the body.

According to an advantageous embodiment, the auxiliary device can include one or more positioning devices that are intended to remain in the body after implantation, and that are formed, at least in sections, or even entirely, of a material that can decompose in the body. The positioning devices can be e.g. bow-shaped, fixed at one end of the body, and dimensioned in terms of length such that the correct positioning of the support frame is ensured when the annulus is contacted.

Auxiliary devices are also feasible that are used to temporarily anchor the support frame and fix it in position until it has adhered. These anchoring devices can have a ring shape with a diameter that exceeds the diameter of the support frame at this position.

In this case, an “end of the support frame” is intended to mean, in particular, a region of the valve implant that is adjoined in only one direction by an implant structure or support frame structure, such as a wire mesh. In this case, a “main extension direction” is intended to mean, in particular, a length of the support frame and/or, in the implanted state of the valve implant, a direction along a flow direction of a flow medium. “At opposite ends” is intended to mean, in particular, ends that are disposed at different ends relative to the flow direction, or at the beginning and at the end of the valve implant. The embodiment according to the invention provides a medical valve implant that is tailored to the parameters of the implantation site, such as an active agent absorption by a cavity wall adjacent to the implant, and/or a flow velocity of a flow medium that flows through the cavity or the implant.

Furthermore, a “valve implant” is intended to mean, in particular, a body that functions as a replacement for a non-return valve, permanently or at least for a longer period of time, when implanted in an animal body and/or human body. Other possibilities that are feasible are all medical valve implants that appear suitable to a person skilled in the art, such as an aortic valve, a pulmonary valve, a mitral valve, or a tricuspid valve; particularly advantageously, an embodiment of the medical implant as a stent, in particular a coronary stent, is provided.

Furthermore, a “support frame” in this context is intended to mean, in particular, a structure such as a wire mesh that substantially imparts a shape and/or form to the implant or stent. In addition, the support frame is preferably composed of an elastic or superelastic material such as a metallic material and/or a combination of a plurality of metallic materials, such as iron, magnesium, nickel, tungsten, titanium, zirconium, niobium, tantalum, zinc, silicon, lithium, sodium, potassium, manganese, and/or any other material that appears reasonable to a person skilled in the art. Another possibility would be a zinc-calcium alloy and/or a material having a memory effect, such as a nickel-titanium alloy and/or a copper-zinc-aluminum alloy, preferably Nitinol. Furthermore, it can be advantageous when the support frame includes, at the least, cobalt and/or chromium, preferably in the form of stainless steel and/or a Cr—Ni—Fe steel—preferably the alloy 316L in this case—or a Co—Cr steel. Using this embodiment, an implant can be provided that results in satisfactory coating results and has good dilatability and advantageous flexibility combined with high stability.

Basically, it would also be feasible, however, for the support frame of the valve implant to be composed at least partially of plastic, a ceramic, and/or a biodegradable material.

Favorably, an optimization of percutaneous valve implants in terms of adhesion behavior and lumen can be attained. When only those regions of the valve implant that are required for implantation are composed, at least in sections, of material that can decompose in the body, the additional advantage is attained that unnecessary irritation of the surrounding tissue caused by regions of this type can be prevented, or the irritation is merely temporary and diminishes over time until it disappears entirely.

In addition to better adhesion of the valve implant in the luminal wall, a larger lumen can be attained at the implantation site since a portion of the valve implant decomposes over time, thereby reducing the volume of the valve implant. It is favorable for the wall thickness of the valve implant to be as small as possible to avoid restricting the lumen and to minimize mechanical irritation of the luminal wall.

To attain accurate positioning of the valve implant, some designs of valve implants include “feelers” that provide the operating surgeon with feedback about when the valve implant bears against the desired site of implantation i.e. the annulus. These feelers are no longer required once the valve implant has expanded, but they remain at the implantation site.

Positioning devices are redundant material that remains in the body and potentially results in irritation caused by mechanical irritation, which promotes calcification over time in the region of the valve implant and often makes it necessary to implant a new valve implant.

Parts of the valve implant that are helpful at the moment of implantation and/or that serve a functional purpose for a limited period of time degrade over time, reduce the volume of the valve implant, and no longer contribute to irritation of the implantation site.

Advantageously, the material can be resorbed by the body.

Polymers that decompose slowly are favorable, for example, such as polydioxanone, polyglycolide, polylactic acid [poly-L-lactide, poly(D,L-lactide), and copolymers, and blends such as poly(L-lactide-co-glycolide), poly(D,L-lactide-co-glycolide), poly(L-lactide-co-D,L-lactide), poly(L-lactide-co-trimethylene carbonate)], poly-ε-caprolactone, di- and triblock copolymers of the above-mentioned lactides with polyethylene glycol, polyhydroxyvalerate, ethylvinylacetate, polyethylene oxide, polyphosphorylcholin, polyhydroxybutyric acid (atactic, isotactic, syndiotactic, and their blends), poly(ortho ester), polyanhydrides, etc.

Rapidly decomposable materials can likewise be used, such as saccharides (alginate, chitosan, levan, hyaluronic acid, and uronides, heparin, dextrane, nitrocellulose, cellulose acetate and derivatives of cellulose, maltodextrin, chondroidin sulphate, carrageenan, etc.), fibrin, albumin, polypeptides and their derivatives, etc.

The decomposable material can also include magnesium. Favorably, entire components can be made of the decomposable material e.g. magnesium, and they therefore dissolve entirely. For example, positioning aids that show the operating surgeon while he is inserting the valve implant that the desired site has been reached can be made entirely of the decomposable material.

As an alternative or in addition thereto, a deposit-inhibiting, in particular calcification-inhibiting, coating can be provided, in particular homocysteinic acid. The risk of a disruption or malfunction of the valve implant can therefore be reduced.

Advantageously, the valve implant can be designed as an aortic valve. An embodiment as a pulmonary valve or an embodiment as a mitral valve is likewise feasible.

Improved clinical results can be attained due to a greater lumen and fewer vascular irritations. Advantageously, a valve implant can be created, in particular an aortic valve, which can adhere in a first step (temporary process), and is then no longer fixed in position by metal but rather by the body's natural tissue. This can be advantageous for a second implantation of a valve implant at the same implantation site, in the case of which the new valve implant is placed in the old valve implant. In addition, in the case of an aortic valve of that type, it can be expected that complications such as disruptions of the mitral valve or the need for a cardiac pacemaker can be reduced.

Advantageously, a deposit-inhibiting, in particular calcification-inhibiting, coating can be provided on the valve implant, in particular homocysteinic acid. In particular, the one or more positioning devices can be provided with the deposit-inhibiting, in particular calcification-inhibiting, coating.

### DESCRIPTION OF THE DRAWINGS

The invention is explained in the following in greater detail with reference to embodiments that are depicted in drawings. They show, in a diagrammatic representation:

FIG. 1 depicting a medical valve implant according to the invention, in the implanted state, and

FIG. 2 providing a schematic depiction of the insertion of an aortic valve at an implantation site.

### DETAILED DESCRIPTION

Elements that are functionally identical or similar-acting are labelled using the same reference numerals in the figures. The figures are schematic depictions of the invention. They do not depict specific parameters of the invention. Furthermore, the figures merely show typical embodiments of the invention and should not limit the invention to the embodiments shown.

Regarding elements in a figure that are not described further, reference is made to the respective description of the elements in preceding figures to avoid unnecessary repetition.

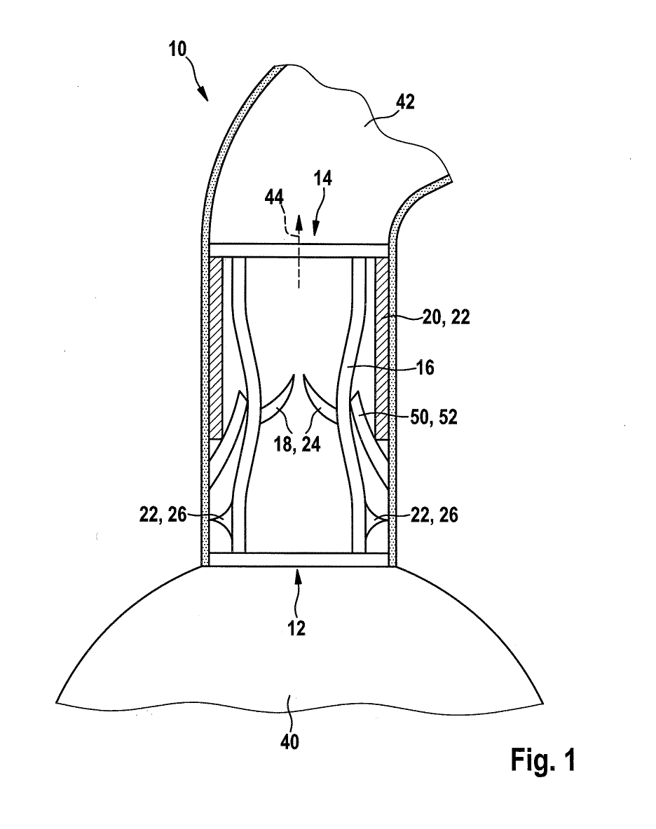

FIG. 1 shows a schematic view of a medical valve implant 10 in the installed state, e.g. in the annulus of an aortic valve 50, that is disposed in a vessel 42, the aorta in this case, in front of left ventricle 40 of the heart.

Valve implant 10 has e.g. the shape of a stent and comprises a support frame 16 that is made e.g. of chromium-cobalt steel and can have, as the support frame structure, a wire mesh, for example, that is formed of stent struts. Furthermore, support frame 16 has a first end 12 and a second end 14 that are disposed on opposite ends of support frame 16 in a main extension direction 44 of support frame 16. In general, support frame 16 can have any shape that appears reasonable to a person skilled in the art.

Support frame 16 is enclosed at its downstream end by a positioning device 20 that contains material 22 that can decompose in the body, and is preferably composed of this material. Support frame 16 presses cusp 52 of natural aortic valve 50 against the luminal wall of vessel 42, thereby creating open space for cusp 18 in the interior of support frame 16. Cusps 18 function as non-return valves and permit blood to flow from ventricle 40 to vessel 42, but block the flow of blood in the opposite direction. Positioning device 20 can be e.g. bow-shaped, fixed at one end of support frame 16, and dimensioned in terms of length such that the correct positioning of support frame 16 is ensured when the annulus is contacted. Positioning device 20 is formed of material 22 that can decompose in the body, and dissolves over time.

Cusps 18 of valve implant 10 can be provided with a coating 24 that prevents the deposition of calcium salts. While positioning device 20 dissolves over time and the lumen of valve implant 10 thereby increases in size, coating 24 prevents a calcification of new cusps 18 of valve implant 10.

A further auxiliary device 26 can be used to temporarily anchor support frame 16 and fix it in position until support frame 16 has adhered. Anchoring devices 26 can have a ring shape with a diameter that exceeds the diameter of support frame 16 at this position. Anchoring devices 26 are formed of material 22 that can decompose in the body, and dissolve over time.

In an embodiment that is not shown in the drawings, in addition or as an alternative, regions of support frame 16 itself can likewise be formed of a material 22 that can decompose in the body.

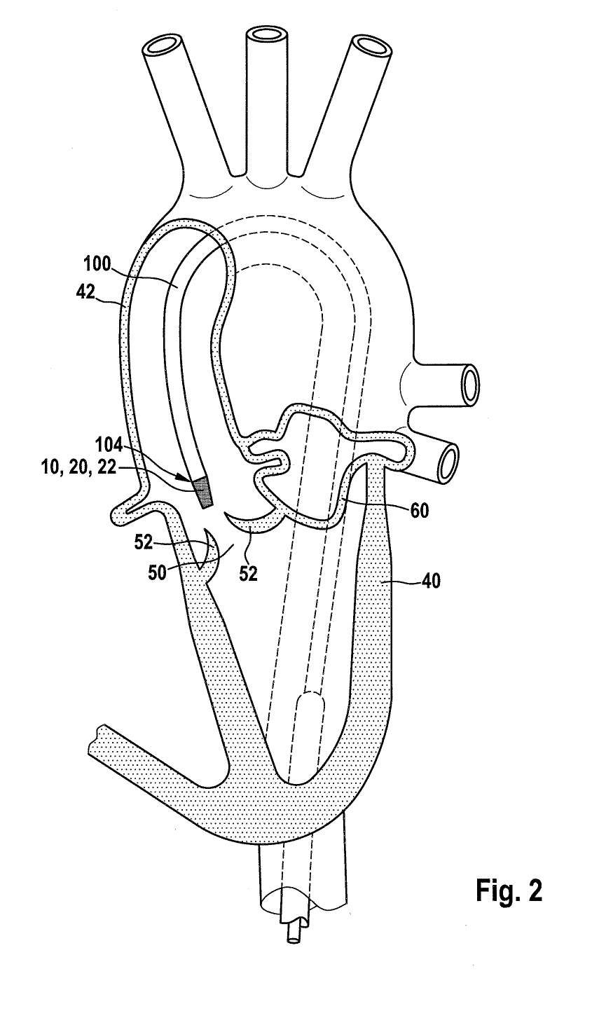

The insertion of medical valve implant 10 is illustrated schematically in a partial sectional view, in FIG. 2. Valve implant 10 is moved, in a compressed state, on a tip 104 of a catheter 100 in a manner known per se to the implantation site e.g. the annulus of aortic valve 50. To ensure that the current position of valve implant 10 can be tracked and valve implant 10 can be positioned at the desired implantation site, a e.g. metallic positioning device 20 is disposed on valve implant 10, which is used to expel valve implant 10 out of catheter 100.

Valve implant 10 is an aortic valve in this embodiment, which can adhere at the implantation site (temporary process) after insertion in a first step. While e.g. metallic positioning device 20 and/or anchoring device 26 dissolve(s) over time, valve implant 10 is then no longer fixed in position by metal, but rather by the body's natural tissue. This is advantageous for a second implantation of a valve implant at the same implantation site, in the case of which the new valve implant is placed in old valve implant 10. Favorably, complications such as disruptions of mitral valve 60 or the need for a cardiac pacemaker can be reduced.

It will be apparent to those skilled in the art that numerous modifications and variations of the described examples and embodiments are possible in light of the above teaching. The disclosed examples and embodiments are presented for purposes of illustration only. Other alternate to embodiments may include some or all of the features disclosed herein. Therefore, it is the intent to cover all such modifications and alternate embodiments as may come within the true scope of this invention.

What is claimed is:

1. A medical valve implant for implantation in the animal body and/or human body, comprising a support frame that includes a first end and a second end, which are disposed on opposite ends of the support frame in a main extension direction of the support frame, characterized in that one or more auxiliary devices that are required for implantation and/or that serve a functional purpose for a limited time and are intended to remain in the body after implantation are formed, at least in sections, of a material that can decompose in the body.

2. The medical valve implant according to claim 1, characterized in that one or more auxiliary devices include at least one positioning device that is intended to remain in the body after implantation and is formed, at least in sections, of a material that can decompose in the body.

3. The medical valve implant according to claim 1, characterized in that one or more auxiliary devices include at least one anchoring device that is intended to remain in the body after implantation and is formed, at least in sections, of a material that can decompose in the body.

4. The medical valve implant according to claim 1, characterized in that the decomposable material includes a polymer selected from the group consisting of polydioxanone, polyglycolide, polylactic acid, poly-ε-caprolactone, and a di- or triblock copolymer;
wherein the polylactic acid is optionally selected from the group consisting of poly-L-lactide, poly(D,L-lactide), and a copolymer or blend thereof optionally selected from the group consisting of poly(L-lactide-co-glycolide), poly(D,L-lactide-co-glycolide), poly(L-lactide-co-D,L-lactide), and poly(L-lactide-co-trimethylene carbonate);
wherein the di- or triblock copolymer comprises an above-listed lactide with polyethylene glycol, polyhydroxyvalerate, ethylvinylacetate, polyethylene oxide, polyphosphorylcholin, polyhydroxybutyric acid (optionally atactic, isotactic, syndiotactic, or a blend thereof), poly(ortho ester), or a polyanhydride.

5. The medical valve implant according to claim 1, characterized in that the decomposable material includes magnesium.

6. The medical valve implant according to claim 1, characterized in that homocysteinic acid is provided as a deposit-inhibiting or calcification inhibiting coating.

7. The medical valve implant according to claim 6, characterized in that the one or more positioning devices is provided with the deposit-inhibiting or calcification-inhibiting coating.

8. The medical valve implant according to claim 1, characterized in that the support frame includes a self-expanding stent.

9. The medical valve implant according to claim 1, characterized in that the support frame includes an elastic and/or superelastic material.

10. The medical valve implant according to claim 1, provided as an aortic valve.

11. The medical valve implant according to claim 1, provided as a pulmonary valve or mitral valve or tricuspid valve.

12. A medical valve implant for implantation in the animal body and/or human body, comprising a support frame that includes a first end and a second end, which are disposed on opposite ends of the support frame in a main extension direction of support frame, characterized in that the support frame is formed, at least in sections, of a material that can decompose in the body.
